(19) United States
(12) Patent Application Publication
(10) Pub. No.: US 2010/0233719 A1
(43) Pub. Date: Sep. 16, 2010

(54) Genetic Markers for Predicting Disease and Treatment Outcome

(75) Inventor: HEINZ-JOSEF LENZ, LOS ANGELES, CA (US)

Correspondence Address: FOLEY & LARDNER LLP, 975 PAGE MILL ROAD, PALO ALTO, CA, 94304, US

(73) Assignee: UNIVERSITY OF SOUTHERN CALIFORNIA

(21) Appl. No.: 12/767,665

(22) Filed: Apr. 26, 2010

Related U.S. Application Data
(63) Continuation of application No. 11/681,708, filed on Mar. 2, 2007.
(60) Provisional application No. 60/779,217, filed on Mar. 3, 2006.

(51) Int. Cl. C12Q 1/68

(52) U.S. Cl. 435/6

(57) ABSTRACT

The present invention provides for a method for identifying patients that are suitably treated by a therapy, such as a therapy involving administration of a fluoropyrimidine drug and/or a platinum drug. The method includes determining the expression level of at least one gene selected from a phospholipase 2 (PLA2) gene, a thymidine phosphorylase (TP) gene, and a glutathione S-transferase P1 (GSTP-1) gene in suitable sample isolated from the patient. Overexpression of the gene or genes identifies the patient as not being suitable for the therapy.

### CROSS-REFERENCE TO RELATED APPLICATIONS

This application claims priority under 35 U.S.C. §119(e) to provisional application No. 60/779,217, filed Mar. 3, 2006, the contents of which are incorporated by reference into the present disclosure.

### FIELD OF THE INVENTION

This invention relates to the field of pharmacogenomics and specifically to the use of genetic markers to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Cancer chemotherapy is limited by the predisposition of specific populations to drug toxicity or poor drug response. Thus, for example, pharmacogenetics (the effect of genetic differences on drug response) has been applied in cancer chemotherapy to understand the significant inter-individual variations in responses and toxicities to the administration of anti-cancer drugs, which may be due to genetic alterations in drug metabolizing enzymes or receptor expression. For a review of the use of germline polymorphisms in clinical oncology, see Lenz, H.-J. (2004) J. Clin. Oncol. 22(13):2519-2521; Park, D. J. et al. (2006) Curr. Opin. Pharma. 6(4):337-344; Zhang, W. et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniqes 39:565-568.

Polymorphism also has been linked to cancer susceptibility (oncogenes, tumor suppressor genes and genes of enzymes involved in metabolic pathways) of individuals. In patients younger than 35 years, several markers for increased cancer risk have been identified. For example, prostate specific antigen (PSA) is used for the early detection of prostate cancer in asymptomatic younger males. Cytochrome P4501A1 and gluthathione S-transferase M1 genotypes influence the risk of developing prostate cancer in younger patients. Similarly, mutations in the tumor suppressor gene, p53, are associated with brain tumors in young adults.

Results from numerous studies suggest several genes may play a major role in the principal pathways of cancer progression and recurrence, and that the corresponding germ-line polymorphisms may lead to significant differences at transcriptional and/or translational levels.

Moreover, while adjuvant chemotherapy and radiation lead to a noticeable improvement in local control among those with cancer, the choice of optimal therapy may be compromised by a wide inter-patient variability of treatment response and host toxicity. Since the rate of inactivation of the administered drug compound may establish its effectiveness in the tumor tissue, genomic variations on different cellular mechanisms that may modify therapy efficacy may influence efficacy.

A number of genes, and/or gene products, have been implicated in the onset and progression of cancer. Among these are genes associated with the processes occurring in the tumor microenvironment including angiogenesis, inter-cellular adhesion, mitogenesis, and inflammation.

Angiogenesis, which involves the formation of capillaries from preexisting vessels, has been characterized by a complex surge of events involving extensive interchange between cells, soluble factors (e.g. cytokines), and extracellular matrix (ECM) components (Balasubramanian (2002) Br. J. Cancer 87:1057). In addition to its fundamental role in reproduction, development, and wound repair, angiogenesis has been shown to be deregulated in cancer formation (Folkman (2002) Semin. Oncol. 29(6):15).

The interleukin family is known to play an important role in the angiogenic process. Interleukin-8 (IL-8), an inflammatory cytokine with angiogenic potential, has been implicated in cancer progression in a variety of cancer types including colorectal carcinoma, glioblastoma, and melanoma (Yuan (2000) Am. J. Respir. Crit. Care Med. 162:1957).

Inter-cellular adhesion plays a major role in both local invasion and metastasis. Cell adhesion molecules (CAMs), which are cell-surface glycoproteins that are crucial for cell-to-cell interactions, have been shown to directly control differentiation, and interruption of normal cell-to-cell contacts has been observed in neoplastic transformation and in metastasis (Edelman (1988) Biochem. 27:3533 and Ruoslahti (1988) Ann. Rev. Biochem. 57:375). Overexpression of ICAM-1 in colorectal cancers has been shown to favor the extravasation and trafficking of cytotoxic lymphocytes toward the neoplastic cells, leading to host defense (Maurer (1998) Int. J. Cancer (Pred. Oncol.) 79:76).

A polymorphism in the gene coding for COX-2 has also been studied. COX-2 is involved in prostaglandin synthesis, and stimulates inflammation and mitogenesis; it has been shown to be markedly overexpressed in colorectal adenomas and adenocarcinomas when compared to normal mucosa (Eberhart (1994) Gastro. 107:1183).

Another family of genes playing a critical role in angiogenesis and tumor progession is the receptor tyrosine kinase family of fibroblast growth factor receptors (FGFRs). FGFRs are also involved in tumor growth and cell migration. The complex pathways of the tumor microenvironment have become the focus of widespread investigation for their role in tumor progression.

Phospholipases A2 (PLA2s) are a large family of enzymes implicated in the angiogenic pathway. PLA2s specifically deacylate fatty acids from the 2nd carbon atom (sn2, thus PLA2) of the triglyceride backbone of phospholipids, producing a free fatty acid and a lyso-phospholipid. PLA2s are ubiquitous enzymes, though the individual enzymes expression patterns differ dramatically (Six and Dennis, (2000) Biochimica et Biophysica Acta. 1488(1-2):1-19).

Differences in drug metabolism, transport, signaling and cellular response pathways also have been shown to collectively influence diversity in patients' reactions to therapy (Evans (1999) Science 286:487). Metabolism of chemotherapeutic agents and radiation-induced products of oxidative stress, therefore, may play a critical role in treatment response. The glutathione s-transferase (GST superfamily) participates in the detoxification processes of platinum compounds (Ban (1996) Cancer Res. 56:3577 and Goto (1999) Free Rad. Res. 31:549). Glutathione S-transferase pi gene (GSTP-I) polymorphism has been associated with response to platinum-based chemotherapy (Stoehlmacher (2002) J. Nat. Cancer Inst. 94:936).

Thymidylate synthase (TS), dihydropyrimidine dehydrogenase (DPD), and thymidine phosphorylase (TP) are important regulatory enzymes involved in the metabolism of the chemotherapeutic drug 5-Fluorouracil (5-FU). TP has been found to be overexpressed in various tumors and plays an important role in angiogenesis, tumor growth, invasion and metastasis (Akiyama, et al., (2004) Cancer Sci. November; 95(11):851-7; Toi, M., et al. (2005) Lancet Oncology, 6:158-166).

Cell cycle regulation provides the foundation for a critical balance between proliferation and cell death, which are important factors in cancer progression. For example, a tumor suppressor gene such as p53 grants the injured cell time to repair its damaged DNA by inducing cell cycle arrest before reinitiating replicative DNA synthesis and/or mitosis (Kastan (1991) Cancer Res. 51:6304). More importantly, when p53 is activated based on DNA damage or other activating factors, it can initiate downstream events leading to apoptosis (Levine (1992) N. Engl. J. Med. 326:1350). The advent of tumor recurrence after radiation therapy depends significantly on how the cell responds to the induced DNA damage; that is, increased p53 function should induce apoptosis in the irradiated cell and thereby prevent proliferation of cancerous cells, whereas decreased p53 function may decrease apoptotic rates.

Finally, DNA repair capacity contributes significantly to the cell's response to chemoradiation treatment (Yanagisawa (1998) Oral Oncol. 34:524). Patient variability in sensitivity to radiotherapy can be attributed to either the amount of damage induced upon radiation exposure or the cell's ability to tolerate and repair the damage (Nunez (1996) Rad. Onc. 39:155). Irradiation can damage DNA directly or indirectly via reactive oxygen species, and the cell has several pathways to repair DNA damage including double-stranded break repair (DSBR), nucleotide excision repair (NER), and base excision repair (BER). An increased ability to repair direct and indirect damage caused by radiation will inherently lower treatment capability and hence may lead to an increase in tumor recurrence. Genes associated with DNA repair include XRCC1 and ERCC2 (Thompson, L. H., (1991) Mutat Res. 247(2):213-9).

Colorectal cancer (CRC) represents the second leading lethal malignancy in the USA. In 2005, an estimated 145,290 new cases will be diagnosed and 56,290 deaths will occur (Jemal, A. et al. (2005) Cancer J. Clin. 55:10-30). Despite advances in the treatment of colorectal cancer, the five year survival rate for metastatic colon cancer is still low, with a median survival of 18-21 months (Douglass, H. O. et al. (1986) N. Eng. J. Med. 315:1294-1295). Accordingly, it is desirable to provide a reliable screening method capable of predicting the clinical outcome of a specific therapeutic regime for treating CRC and other related gastrointestinal cancers.

### DESCRIPTION OF THE EMBODIMENTS

This invention provides methods for selecting a therapeutic regimen or determining if a certain therapeutic regimen is more likely to treat a cancer or is the appropriate chemotherapy for that patient than other available chemotherapies.

One aspect is a method for identifying patients suffering from a gastrointestinal cancer and that are suitably treated by a therapy by determining the expression level of at least one gene selected from the group consisting of phospholipase 2 (PLA2) gene, thymidine phosphorylase (TP) gene, and glutathione S-transferase P1 (GSTP-1) gene, in suitable sample isolated from the patient. If the sample indicates overexpression of the gene(s) then that patient should not receive a therapy identified below. In one embodiment, the expression level of at least two of these genes are determined. In another embodiment, the expression level of phospholipase 2 (PLA2) gene, thymidine phosphorylase (TP) gene, and glutathione S-transferase P1 (GSTP-1) gene are determined. In yet a further embodiment, only the expression level of phospholipase 2 (PLA2) gene is determined. The expression levels of the genes are compared to an internal control, such as the β-actin gene, to identify those genes that are overexpressed.

In another aspect, the patient is suffering from a solid malignant tumor such as a gastrointestinal tumor, e.g., from rectal cancer, colorectal cancer, metastatic colorectal cancer, colon cancer, gastric cancer, lung cancer, non-small cell lung cancer and esophageal cancer. In an alternative aspect, the patient is suffering from colorectal cancer.

In an alternative embodiment, the expression level of COX-2 gene is determined in the sample individually or in addition to determining the expression level of at least one gene selected from the group consisting of phospholipase 2 (PLA2) gene, thymidine phosphorylase (TP) gene, and glutathione S-transferase P1 (GSTP-1) gene. If the COX-2 gene is underexpressed as compared to expression in the control, then the patient should not receive therapy comprising administration of a fluoropyrimidine drug and a platinum drug.

The therapy under consideration comprises administration of at least one of a fluoropyrimidine drug and a platinum drug, or equivalents thereof. In one embodiment, the fluoropyrimidine drug is 5-FU and the platinum drug is oxaliplatin, or equivalents thereof.

Another aspect of the invention is a method for identifying patients that are at risk for undesirable side effects or those not likely to benefit from a pre-selected therapy. The method comprises determining the expression level of at least one gene selected from the group consisting of XRCC1 gene and IL-8 gene in suitable sample isolated from the patient, wherein overexpression of the gene(s) identifies the patient as being at a risk for undesirable side effects. In one embodiment of this aspect, the expression level of both XRCC1 gene and IL-8 gene is determined. In another embodiment, the side effect is toxicity. In a yet a further aspect, overexpression of the genes indicates that administration of the treatment is not likely to enhance progression-free survival from date of administration of the therapy.

The therapy under consideration comprises administration of at least one of a fluoropyrimidine drug and a platinum drug, or equivalents thereof. In one embodiment, the fluoropyrimidine drug is 5-FU and the platinum drug is oxaliplatin, or equivalents thereof.

The suitable sample used in the above described methods is at least one of a tumor sample, a sample of normal tissue corresponding to the tumor sample and a peripheral blood lymphocyte. In one aspect, the method also requires isolating a sample containing the genetic material to be tested from the patient; however, it is conceivable that one of skill in the art will be able to analyze and identify genetic polymorphisms in situ at some point in the future. Accordingly, the inventions of this application are not to be limited to requiring isolation of the genetic material prior to analysis.

These methods are not limited by the technique that is used to identify the expression level of the gene of interest. Methods for measuring gene expression are well known in the art and include, but are not limited to, immunological assays, nuclease protection assays, northern blots, in situ hybridization, and Real-Time Polymerase Chain Reaction (RT-PCR), expressed sequence tag (EST) sequencing, cDNA microarray hybridization or gene chip analysis, subtractive cloning, Serial Analysis of Gene Expression (SAGE), Massively Parallel Signature Sequencing (MPSS), and Sequencing-By-Synthesis (SBS).

After a patient has been identified as positive and therefore not suitable for the therapy, the method may further comprise administering or delivering an effective amount of therapy that excludes administration of a fluoropyrimidine and/or a platinum drug or biological equivalents thereof. Methods of administration of pharmaceuticals and biologicals are known in the art and incorporated herein by reference.

This invention also provides a kit, software and/or gene chip for patient sampling and performance of the methods of this invention. The kits contain gene chips, software, probes or primers that can be used to determine the expression level of the gene of interest. In an alternate embodiment, the kit contains antibodies or other polypeptide binding agents to can be used to quantify the expression level of the gene of interest. Instructions for using the materials to carry out the methods are further provided.

It will be appreciated by one of skill in the art that the embodiments summarized above may be used together in any suitable combination to generate additional embodiments not expressly recited above, and that such embodiments are considered to be part of the present invention

### MODES FOR CARRYING OUT THE INVENTION

The present invention provides methods and kits for determining a patient's likely response to specific cancer treatment by determining the patient's genotype at a gene of interest and/or the level of expression of a gene of interest. Other aspects of the invention are described below or will be apparent to one of skill in the art in light of the present disclosure.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook et al. MOLECULAR CLONING: A LABORATORY MANUAL, 2nd edition (1989); CURRENT PROTOCOLS IN MOLECULAR BIOLOGY (F. M. Ausubel et al. eds. (1987)); the series METHODS IN ENZYMOLOGY (Academic Press, Inc., N.Y.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); ANIMAL CELL CULTURE (R. I. Freshney ed. (1987)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); IMMUNOCHEMICAL METHODS IN CELL AND MOLECULAR BIOLOGY (Mayer and Walker, eds., Academic Press, London (1987)); HANDBOOK OF EXPERIMENTAL IMMUNOLOGY, Volumes I-IV (D. M. Weir and C. C. Blackwell, eds. (1986)); MANIPULATING THE MOUSE EMBRYO (Cold Spring Harbor Laboratory Press, Cold Spring Harbor, N.Y. (1986)).

Definitions

As used herein, certain terms may have the following defined meanings As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the compositions and methods. Thus, a composition consisting essentially of the elements as defined herein would not exclude trace contaminants from the isolation and purification method and pharmaceutically acceptable carriers, such as phosphate buffered saline, preservatives, and the like. “Consisting of shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “vector” refers to a nucleic acid molecule capable of transporting another nucleic acid to which it has been linked. One type of preferred vector is an episome, i.e., a nucleic acid capable of extrachromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes. The term “allelic variant of a polymorphic region of the gene of interest” refers to a region of the gene of interest having one of a plurality of nucleotide sequences found in that region of the gene in other individuals.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype' refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “gene of interest” intends one or more genes selected from the group consisting of thymidine phosphorylase (TP) gene, XRCC1 gene, COX-2 gene, IL-8 gene, phospholipase 2 (PLA2) gene, and glutathione S-transferase P1 (GSTP-1) gene.

An expression “database” denotes a set of stored data that represent a collection of sequences, which in turn represent a collection of biological reference materials.

The term “cDNAs” refers to complementary DNA, that is mRNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

“Differentially expressed” as applied to a gene, refers to the differential production of the mRNA transcribed from the gene or the protein product encoded by the gene. A differentially expressed gene may be overexpressed or underexpressed as compared to the expression level of a normal or control cell or with an internal control. In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample. The term “differentially expressed” also refers to nucleotide sequences in a cell or tissue which are expressed where silent in a control cell or not expressed where expressed in a control cell.

A “control” is used in an experiment for comparison or normalization purposes. A control can be positive or negative. Controls for use in comparing gene expression at the mRNA level include internal and external controls. An internal control refers to a gene known to be present in the sample to be tested. The expression level of the gene is preferably well characterized and provides a reliable measure of gene expression level in the control. Examples of genes that are useful as internal controls include, but are not limited to, housekeeping genes such as β-actin, 18S, glyceraldehyde-3-phosphate dehydrogenase (GAPDH), and cyclophilin. External controls include use of a subject or a sample from a subject, known to express the gene of interest a certain level.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to a patient sample refers to tissue, cells, genetic material and nucleic acids, such as DNA or RNA, separated from other cells or tissue or DNAs or RNAs, respectively, that are present in the natural source. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “mismatches” refers to hybridized nucleic acid duplexes which are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, efc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

A “polymorphic gene” refers to a gene having at least one polymorphic region.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

The antibodies can be polyclonal or monoclonal and can be isolated from any suitable biological source, e.g., murine, rat, sheep and canine

The term “treating” or “treats” as used herein is intended to encompass curing as well as ameliorating at least one symptom of the condition or disease. For example, in the case of cancer, treatment includes a reduction in cachexia, increase in survival time, elongation in time to tumor progression, reduction in tumor mass, reduction in tumor burden and/or a prolongation in time to tumor metastasis, each as measured by standards set by the National Cancer Institute and the U.S. Food and Drug Administration for the approval of new drugs. See Johnson et al. (2003) J. Clin. Oncol. 21(7):1404-1411.

A “suitable therapy” as used herein implies treatment with a fluoropyrimidine drug and/or a platinum drug. In one embodiment, a suitable therapy is treatment with 5-FU and oxiliplatin.

An “undesirable side effect” refers to unwanted, negative consequences associated with a therapy. For example, undesirable side effects include an increase in the risk of toxicity, medical or physiological complications that negatively affect the patient's prognosis, and pathological changes occurring at the cellular or subcellular level. In one embodiment, the undesirable side effect is an increase in the risk of toxicity.

“Toxicity” is evaluated as discussed in the Common Toxicity Criteria Manual, Version 2.0, Jun. 1, 1999, National Cancer Institute. In one embodiment, the toxicity is a cumulative grade 2+ or higher.

A “response” implies a measurable reduction in tumor size or evidence of disease.

A “complete response” (CR) to a therapy defines patients with evaluable but non-measurable disease, whose tumor and all evidence of disease had disappeared.

A “partial response” (PR) to a therapy defines patients with anything less than complete response were simply categorized as demonstrating partial response. Clinical parameters include those identified above.

“Non-response” (NR) to a therapy defines patients whose tumor or evidence of disease has remained constant or has progressed.

“Stable disease” (SD) indicates that the patient is stable.

“Overall Survival” (OS) intends a prolongation in life expectancy as compared to naïve or untreated individuals or patients.

“Time to tumor progression” is the time between treatment and initial response and the time when resistance to initial treatment or loss of treatment efficacy.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat a gastrointestinal cancer.

The compounds can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

This invention identifies cancer patients that may be treated by administration of a therapy comprising administration of a fluoropyrimidine drug such as 5-FU alone or in combination with a platinum drug, such as oxaliplatin. It also provides a method for determining if a certain therapeutic regimen is more likely to treat a cancer or present undesirable side effects and therefore, is the appropriate chemotherapy for that cancer patient than other available chemotherapies.

The methods are useful for patients suffering from a cancer or neoplasm that is treatable by use of one or more of platinum-based therapy (oxaliplatin, cisplatin, carboplatin) fluropyrimidine-based therapy (5-fluorouracil (5-FU), floxuriden (FUDR) capecitabine, UFT), irinotecan (CP-11), radiation and surgical resection. Non-limiting examples of such cancers include, but are not limited to, gastrointestinal (GI) cancers such as rectal cancer, colorectal cancer, colon cancer, gastric cancer, lung cancer, and non-small lung cancer (NSCLC) and esophageal cancer. In one embodiment, the cancer comprises advanced colorectal cancer (CRC) that may be treatable with fluoropyrimidine drug and a platinum drug, or their equivalents, or combinations thereof. In a another embodiment, the fluoropyrimidine drug is 5-FU and the platinum drug is oxaliplatin, or equivalents thereof.

5-FU (5-fluorouracil) is an antimetabolite drug that has been in use for over four decades. It targets thymidylate synthase and the enzyme dihydorpyrimidine dehydrogenase (DPD). Several derivatives and substitutes for 5-FU and their use in gastric cancer have been reported in Ajani (2005) The Oncologist 10 (suppl.3):49-58. It is often used in combination with the platinum drug oxaliplatin and irinotecan.

Oxaliplatin is a relatively new diammine cyclohexane platinum derivative that is active in several solid tumor types, especially in some cisplatin/carboplatin refractory diseases such as colorectal cancer (Machover et al. (1996) Ann. Oncol. 7:95-98) and is reported to be better tolerated than cisplatin, especially in terms of renal toxicity. Grolleau, F. et al. (2001) supra.

In one embodiment, the chemotherapeutic regimen further comprises radiation therapy. In an alternate embodiment, the therapy comprises administration of an antibody, such as an anti-VEGF antibody, such as Avastin, or a biological equivalent of the antibody.

The Applicant has determined that high levels of expression of phospholipase 2 (PLA2) gene, thymidine phosphorylase (TP) gene, and glutathione S-transferase P1 (GSTP-1) gene, for example, in the tumor cells of GI cancer patients treated with a combination therapy of a fluoropyrimidine drug, such as 5-FU, and a platinum drug, such as oxaliplatin, correlates to a decrease in overall survival rate. There is also a trend in the association between high mRNA levels of PLA2 and shorter progression free survival in GI cancer patients undergoing the combination therapy. The correlations indicate that those patients that overexpress these genes will not benefit from the combination therapy and therefore would not be suitably treated by the combination of 5-FU and oxaliplatin. Other therapies should therefore be pursued for these patients.

Accordingly, one aspect of this invention is a method to identify patients that are not suitable candidates for administration of the above-noted therapies. The expression level of at least one gene selected from the group consisting of phospholipase 2 (PLA2), thymidine phosphorylase (TP) gene, and glutathione S-transferase P1 (GSTP-1) gene is determined in suitable sample isolated from the patient. If the patient sample indicates overexpression of the gene(s), use of this therapy should not be utilized for this patient. Alternate embodiments of the method include determining the expression level of at least two of the genes, and determining the expression level of all of the genes. In an alternate embodiment, the expression level of at least PLA 2 also is determined.

The methods of the invention are applicable to therapies comprising administration of at least one fluoropyrimidine drug, or equivalent thereof, alone or in combination with at least one platinum drug, or equivalent thereof. In an alternate embodiment, the therapy comprises administration of 5-FU and oxaliplatin, or equivalents thereof.

Applicant has further determined that low levels of expression of COX-2 gene, for example, in the tumor cells isolated from a GI cancer patient treated with a combination therapy of a fluoropyrimidine, such as 5-FU, and a platinum drug, such as oxaliplatin, correlates to a decrease in overall survival rate. The correlation indicates that those patients that underexpress COX-2 gene will not benefit from the combination therapy and therefore would not be suitably treated by the combination. Thus, a patient diagnosed with a GI cancer with a tumor sample that underexpresses COX-2 gene is unlikely to respond to this therapy and alternative therapies should be selected.

Applicant has also determined that high levels of expression of XRCC 1 gene and IL-8 gene in patient samples treated with a combination therapy of a fluoropyrimidine and a platinum drug, e.g., 5-FU and oxaliplatin, correlates to an increase in side effects from the combination therapy as compared to patients who did not overexpress these genes. Side effects include an increase in the risk of cumulative grade 3+ toxicity. The correlations indicate that those patients that overexpress these genes would not be suitably treated by this therapy. This information may be useful, for example, for selecting alternative therapies and/or for dosing modification as well for identifying patients at high risk for serious side effects.

The methods of the invention requires screening of a sample from a patient to determine the expression level of the gene(s). In one embodiment, the sample to be screened is the tumor tissue itself or normal tissue immediately adjacent to the tumor. In a further embodiment, the sample is of normal tissue corresponding to the tumor sample. In yet a further embodiment, any cell expected to carry the gene of interest, when the polymorphism is genetic, such as a peripheral blood lymphocyte.

Diagnostic Methods

The invention further features predictive medicines, which are based, at least in part, on determination of the expression level of the gene of interest.

For example, information obtained using the diagnostic assays described herein is useful for determining if a patient will respond to cancer treatment of a given type or present undesirable side effects. Based on the prognostic information, a doctor can recommend a regimen or therapeutic protocol, useful for treating cancer in the individual.

In addition, this knowledge allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug; and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup specific; and 3) an accelerated and less costly development for drug candidates and more optimal drug labeling.

The methods of the present invention are directed to determining expression levels and/or differential expression of the genes of interest identified herein. These methods are not limited by the technique that is used to identify the expression level of the gene of interest. Methods for measuring gene expression are well known in the art and include, but are not limited to, immunological assays, nuclease protection assays, northern blots, in situ hybridization, and Real-Time Polymerase Chain Reaction (RT-PCR), expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli, et al., (1998) J. Cell. Biochem. 72 (S30-31): 286-296; Galante, P. A. F., et al., (2007) Bioinformatics, Advance Access (Feb. 3, 2007), both of which are incorporated by reference herein

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. Velculescu, V. E. et al., (1995) Science, 270 (5235):484-487; Velculescu, V. E., (1997) Cell 88(2):243-251, both of which are incorporated by reference herein.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. Brenner, et al. (2000) Nat. Biotechnol. 18:630-634; Reinartz, J., et al., (2002) Brief Funct. Genomic Proteomic 1: 95-104, both of which are incorporated by reference herein.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon, K., et al., (2002) PNAS 99:11287-92), SAGEmap (Lash et al., (2000) Genome Res 10:1051-1060), and Automatic Correspondence of Tags and Genes (ACTG) (Galante, (2007)). The data can also be analyzed using databases constructed using in house computers (Blackshaw, et al. (2004) PLoS Biol, 2:E247; Silva, et al., (2004) Nucleic Acids Res 32: 6104-6110)).

Over- or underexpression of a gene, in some cases, is correlated with a genomic polymorphism. The polymorphism can be present in a open reading frame (coded) region of the gene, in a “silent” region of the gene, in the promoter region, or in the 3′ untranslated region of the transcript. Methods for determining polymorphisms are well known in the art and include, but are not limited to, the methods discussed below.

Detection of point mutations can be accomplished by molecular cloning of the specified allele and subsequent sequencing of that allele using techniques known in the art. Alternatively, the gene sequences can be amplified directly from a genomic DNA preparation from the tumor tissue using PCR, and the sequence composition is determined from the amplified product. As described more fully below, numerous methods are available for analyzing a subject's DNA for mutations at a given genetic locus such as the gene of interest.

Another detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (Genechip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bioflechnology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert ((1997) Proc. Natl Acad Sci, USA 74:560) or Sanger (Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number W094/16101, entitled DNA Sequencing by Mass Spectrometry by H. Koster; U.S. Pat. No. 5,547,835 and Internationa Patent Application Publication Number WO 94/21822 entitled “DNA Sequencing by Mass Spectrometry Via Exonuclease Degradation” by H. Koster; U.S. Pat. No. 5,605,798 and International Patent Application No. PCT1US96103651 entitled DNA Diagnostics Based on Mass Spectrometry by H. Koster; Cohen et al. (1996) Adv. Chromat. 36:127-162; and Griffin et al. (1993) Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method Of DNA Sequencing Employing A Mixed DNA Polymer Chain Probe” and U.S. Pat. No. 5,571,676 entitled “Method For Mismatch-Directed In Vitro DNA Sequencing.”.

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265: 1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl Acad. Sci USA 86:6230 and Wallace et al. (1979) Nucl. Acids Res. 6:3543). Such allele specific oligonucleotide hybridization techniques may be used for the detection of the nucleotide changes in the polylmorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al. Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

Other methods include a solution-based method for determining the identity of the nucleotide of the polymorphic site. Cohen, D. et al. (French Patent 2,650,840; PCT Appln. No. W091/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. Publication No. W092/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. Publication No. W091/02087) the method of Goelet, P. et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al. (1989) Nucl. Acids. Res. 77:7779-7784; Sokolov, B. P. (1990) Nucl. Acids Res. 18:3671; Syvanen, A.-C., et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:I 59-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al., (1989) supra, at Chapter 18. The protein detection and isolation methods employed herein can also be such as those described in Harlow and Lane, (1988) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

Moreover, it will be understood that any of the above methods for detecting alterations in a gene or gene product expression or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a patient has or is at risk of developing disease such as colorectal cancer.

Sample nucleic acid for use in the above-described diagnostic and prognostic methods can be obtained from any cell type or tissue of a patient. For example, a patient's bodily fluid (e.g. blood) can be obtained by known techniques (e.g., venipuncture). Alternatively, nucleic acid tests can be performed on dry samples (e.g., hair or skin) Fetal nucleic acid samples can be obtained from maternal blood as described in International Patent Application Publication No. WO91/07660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures, see, for example, Nuovo, G. J. (1992) “PCR In Situ Hybridization: Protocols And Applications”, Raven Press, NY.

Nucleic Acids

In one aspect, the nucleic acid sequences of the gene's allelic variants, or portions thereof, can be the basis for probes or primers, e.g., in methods for determining the expression level of the gene. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the region of interest and which are not further extended. For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double-stranded nucleic acid, such that upon extension from each primer, a double-stranded nucleic acid is amplified.

Yet other preferred primers of the invention are nucleic acids which are capable of selectively hybridizing to an allelic variant of a polymorphic region of the gene of interest. Thus, such primers can be specific for the gene of interest sequence, so long as they have a nucleotide sequence which is capable of hybridizing to the gene of interest.

The probe or primer may further comprise a label attached thereto, which, e.g., is capable of being detected, e.g. the label group is selected from amongst radioisotopes, fluorescent compounds, enzymes, and enzyme cofactors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization triggered cleavage agents, (see, e.g., Krol et al., (1988) BioTechniques 6:958-976) or intercalating agents (see, e.g., Zon (1988) Pharm. Res. 5:539-549). To this end, the nucleic acid used in the methods of the invention may be conjugated to another molecule, e.g., a peptide, hybridization triggered crosslinking agent, transport agent, hybridization-triggered cleavage agent, etc. The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (1989) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451).

Methods of Treatment

The invention further provides methods of treating subjects suffering from gastrointestinal cancer after determining the expression level of the genes of interest. Patients that do not overexpress these genes or underexpress COX-2, are suitable for therapy that includes administering an effective amount of one or more of a fluoropyrimidine drug and/or a platinum drug, or equivalents thereof. In one embodiment, the fluoropyrimidine drug is 5-FU and the platinum drug is oxaliplatin. In an alternate embodiment, the method comprises (a) determining the expression level of a predetermined gene as identified herein as relevant to treatment with a fluoropyrimidine drug and/or a platinum drug, or equivalents thereof; and (b) administering to a subject that does not overexpress or underexpress the genes of interest, an effective amount of one or more of a fluoropyrimidine drug or a platinum drug, or equivalents thereof. In a preferred embodiment, the fluoropyrimidine drug is 5-FU and the platinum drug is oxaliplatin.

### Kits

As set forth herein, the invention provides diagnostic methods for determining the expression level of a gene of interest, or the type of allelic variant of a polymorphic region present in the gene of interest. In some embodiments, the methods use probes or primers comprising nucleotide sequences which are complementary gene of interest or to the polymorphic region of the gene of interest. Accordingly, the invention provides kits for performing these methods.

The invention further provides a kit for determining whether a subject is likely to respond to respond to therapy comprising administration of at least one of a fluoropyrimidine drug or a platinum drug, or equivalents thereof. In a preferred embodiment, the fluoropyrimidine drug is 5-FU, and the platinum drug is oxaliplatin.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the gene of interest. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) “An Introduction to Radioimmunoassay and Related Techniques” Elsevier Science Publishers, Amsterdam, The Netherlands; Bullock, G. R. et al., “Techniques in Immunocytochemistry” Academic Press, Orlando, Fla. Vol. 1 (1982), Vol. 2 (1983), Vol. 3 (1985); Tijssen, P., (1985) “Practice and Theory of Immunoassays: Laboratory Techniques in Biochemistry and Molecular Biology”, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the expression level of a gene of interest or a patient's genotype in the polymorphic region of a gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the gene of interest can also be useful for identifying an individual among other individuals from the same species. For example, DNA sequences can be used as a fingerprint for detection of different individuals within the same species (Thompson, J. S. and Thompson, eds., (1991) “Genetics in Medicine”, W B Saunders Co., Philadelphia, Pa.). This is useful, e.g., in forensic studies.

The invention now being generally described, it will be more readily understood by reference to the following examples which are included merely for purposes of illustration of certain aspects and embodiments of the present invention, and are not intended to limit the invention.

### Experimental Example

Analysis of Intratumoral mRNA Levels to Predict Clinical Outcome

This study investigated whether mRNA expression levels of thymidine phosphorylase (TP), XRCC1, COX-2, IL-8, phospholipase 2 (PLA2)), and glutathione S-transferase P1 (GSTP-1) are associated with the clinical outcome in patients with metastatic colorectal cancer (CRC) treated with 5-fluorouracil (5-FU) and oxaliplatin. Overall survival was the primary endpoint. Progression-free survival, response, and toxicity were the secondary endpoints.

Patients

Eighty-five patients with metastatic CRC treated with second-line 5-FU/Oxaliplatin from a prospectively followed cohort of patients were included in this study.

Sample Preparation and Analysis

Quantitation of gene expression can be performed by any method known in the art. For the purpose of illustration only the following example is provided.

Intratumoral mRNA levels is assessed from paraffin-embedded tissue samples using laser capture microdissection and quantitative Real-time PCR as discussed below. For the evaluation of mRNA levels in metastatic colorectal cancer, tumor samples are obtained from the primary colorectal tumor or from metastatic site of the liver at the time of diagnosis. Paraffin-embedded tumor blocks are reviewed for quality and tumor content by a pathologist. Ten (10) micrometer thick sections are obtained from the identified areas with the highest tumor concentration. Sections are mounted on uncoated glass slides. For histology diagnosis, three representative sections, consisting of the beginning, the middle and the end of sections of the tissue are stained with H&E by the standard method. Before microdissection, sections are deparafinized in xylene for 10 minutes and hydrated with 100%, 95% and finally 70% ethanol. Then they are washed in H₂0 for 30 seconds. Afterwards, they are stained with nuclear fast red (NFR, American MasterTech Scientific, Inc.) for 20 seconds and rinsed in H₂0 for 30 seconds. Samples are then dehydrated with 70% ethanol, 95% ethanol and 100% ethanol for 30 seconds each, followed by xylene for 10 minutes. The slides are then completely air-dried. If the histology of the samples is homogeneous and contain more than 90% tissue of interest, samples are dissected from the slides using a scalpel. All other sections of interest are selectively isolated by laser capture microdissection (P.A.L.M. Microsystem, Leica, Wetzlar, Germany) according to the standard procedure. The dissected particles of tissue are transferred to a reaction tube containing 400 microliters of RNA lysis buffer.

RNA isolation from paraffin-embedded samples is done according to a proprietary procedure of Response Genetics, Inc. (Los Angeles, Calif.; U.S. Pat. No. 6,248,535). cDNA is prepared as described in Lord, R. V. et al. (2000) J. Gastrointest. Surg. 4:135-142.

Quantitation of gene of interest and an internal reference gene, beta-actin, is done using a fluorescence based real-time detection method (ABI PRISM 7900 Sequence detection System (TAGMAN®) Perkin-Elmer (PE) Applied Biosystem, Foster City, Calif., USA). The PCR reaction mixture consists of 1200 nM of each primer, 200 nM probe, 0.4 U of AmpliTaq Gold Polymerase, 200 nM each dATP, dCTP, dGTP, dTTP, 3.5 mM 20 MgCl2 and 1× Taqman Buffer A containing a reference dye, to a final volume of 20 microliter (all reagents from PE Applied Biosystems, Foster City, Calif., USA). Cycling conditions are 50° C. for 2 min, 95° C. for 10 min, followed by 46 cycles at 95° C. for 15 s and 60° C. for 1 min. The primers and probes to be used are based on the sequence of specific gene or genes analyzed in the experiment. Table 1 provides a list of the primers and probes useful in quantitation of gene expression. Other probes can be designed by those of skill in the are using the sequence of the target gene.

TABLE 1
Primers and Probes

| | Gen Bank | Forward Primer | Reverse Primer | Taqman Probe |
|---|---|---|---|---|
| Gene | Accession | (5′-3′) | (5′-3′) | (5′-3′) |
| Beta- | NM_001101 | GAGCGCGGCTACAGCTT | TCCTTAATGTCACGCACGATTT | ACCACCACGGCCGAGCGG |
| actin | | (SEQ ID NO: 1) | (SEQ ID NO: 2) | (SEQ ID NO: 3) |
| Cox-2 | NM_000963 | GCTCAACATGATGTTTG | GCTGGCCCTCGCTTATGA | TGCCCAGCACTTCACGCA |
| | | CATTC | (SEQ ID NO: 5) | TCAGTT |
| | | (SEQ ID NO: 4) | | (SEQ ID NO: 6) |
| GSTP-1 | NM_000852 | CCTGTACCAGTCCAATA | TCCTGCTGGTCCTTCCCATA | TCACCTGGGCCGCACCCT |
| | | CCATCCT | (SEQ ID NO: 8) | TG |
| | | (SEQ ID NO: 7) | | (SEQ ID NO: 9) |
| IL-8 | NM_000584 | CAGCTCTGTGTGAAGGT | GGGTGGAAAGGTTTGGAGTATG | TGCACTGACATCTAAGTT |
| | | GCAGTT | TC | CTTTAGCACTCCTTGGC |
| | | (SEQ ID NO: 10) | (SEQ ID NO: 11) | (SEQ ID NO: 12) |
| PLA2 | | CCTACGTTGCTGGTCTT | CTCCTCTGGCCCTTTCTCTG | CCACCTGGTATATGTCAA |
| | | TCTG | (SEQ ID NO: 14) | CCTTGTATTCTCACCC |
| | | (SEQ ID NO: 13) | | (SEQ ID NO: 15) |
| TP | NM_001953 | CCTGCGGACGGAATCCT | GCTGTGATGAGTGGCAGGCT | CAGCCAGAGATGTGACAG |
| | | (SEQ ID NO: 16) | (SEQ ID NO: 18) | CCACCGT |
| | | GAGTGAGCAGCTGGTTC | TGATGAGTGGCAGGCTGTC | (SEQ ID NO: 20) |
| | | CT | (SEQ ID NO: 19) | |
| | | (SEQ ID NO: 17) | | |
| XRCC1 | | CTGGGACCGGGTCAAA | CCGTACAAAACTCAAGCCAAAG | TGCAGCCAGCCCTACAGC |
| | | ATTG | G | AAGGACT |
| | | (SEQ ID NO: 21) | (SEQ ID NO: 22) | (SEQ ID NO: 23) |

TAGMAN® measurements yield Ct values that are inversely proportional to the amount of cDNA in the tube, i.e., a higher Ct value means it requires more PCR cycles to reach a certain level of detection.

Gene expression values (relative mRNA levels) are expressed as ratios (differences between the Ct values) between the gene of interest and an internal reference gene (beta-actin) that provides a normalization factor for the amount of RNA isolated from a specimen.

Results

A total of 85 patients were enrolled in this study, including 40 women and 45 men with a median age of 60 years (range 29-87). The median survival time was 9.7 months with a median progression free survival (PFS) of 4.2 months. 1 (1%) patient had a complete response (CR), 15 (18%) had a partial response (PR), 36 (43%) had a stable disease (SD), and 32 (38%) had a progressive disease (PD).

The results indicate that high intratumoral mRNA levels of PLA2, TP, GSTP-1 and low mRNA levels of COX-2 were each significantly associated with shorter overall survival (P≦0.05, log-rank test). This result indicates that patients with CRC tumors with high levels of expression of PLA2, TP, GSTP-1 and low levels of expression of COX-2 are not suitably treated by a combination therapy comprising fluoropyrimidine and oxaliplatin.

A trend in the association between high mRNA levels of PLA2 and shorter progression-free survival (P=0.08) was detected by this experiment.

In addition, high mRNA levels of XRCC1 and IL-8 were each significantly associated with high risk of cumulative grade 3+ toxicity (P≦0.05).

The study indicated that no significant association exists between intratumoral mRNA expression levels of TP, XRCC1, COX-2, IL-8, PLA2, and GSTP-1 and positive response to the combination therapy.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

We claim:

1. A method for determining whether a patient suffering from a gastrointestinal (GI) cancer is suitably treated by a therapy comprising the administration of a fluoropyrimidine or a platinum drug, the method comprising determining the expression level of at least one gene selected from the group consisting of phospholipase 2 (PLA2) gene, thymidine phosphorylase (TP) gene, and glutathione S-transferase P1 (GSTP-1) gene, in suitable sample isolated from the patient, wherein overexpression of the gene(s) identifies the patient as not suitable for the therapy.

2. The method of claim 1, wherein the method comprises determining the expression level of at least two of the genes.

3. The method of claim 1, wherein the method comprises determining the expression level of phospholipase 2 (PLA2) gene, thymidine phosphorylase (TP) gene, and glutathione S-transferase P1 (GSTP-1) gene.

4. The method of claim 1, wherein the method comprises determining the expression level of the phospholipase 2 (PLA2) gene.

5. The method of claim 1, wherein the therapy comprises administration of at least one of a fluoropyrimidine drug and a platinum drug.

6. The method of claim 5, wherein the fluoropyrimidine drug is 5-FU and the platinum drug is oxaliplatin.

7. The method of claim 1, wherein the suitable sample is at least one of a GI tumor sample, a sample of normal tissue corresponding to the GI tumor sample and a peripheral blood lymphocyte.

8. The method of claim 1, wherein the method further comprises determining the expression level of COX-2 gene in the suitable sample, and wherein underexpression of the COX-2 gene identifies the patient as not suitable for the therapy.

9. The method of claim 8, wherein the therapy comprises administration of a fluoropyrimidine drug and a platinum drug.

10. The method of claim 8, wherein the fluoropyrimidine drug is 5-FU and the platinum drug is oxaliplatin.

11. The method of claim 8, wherein the suitable sample is at least one of a GI tumor sample, a sample of normal tissue corresponding to the GI tumor sample and a peripheral blood lymphocyte.

12. The method of claim 1, wherein the gastrointestinal cancer is selected from the group consisting of rectal cancer, colorectal cancer, metastatic colorectal cancer, colon cancer, gastric cancer, lung cancer, non-small cell lung cancer and esophageal cancer.

13. The method of claim 1, wherein the gastrointestinal cancer is colorectal cancer.

14. The method of claim 8, wherein the gastrointestinal cancer is colorectal cancer.

15. A method for identifying patients suffering from a gastrointestinal cancer that are at risk for suffering from undesirable side effects from administration of a fluoropyrimidine drug and a platinum drug, comprising determining the expression level of at least one gene selected from the group consisting of XRCC1 gene and IL-8 gene in suitable sample isolated from the patient, wherein overexpression of the gene(s) identifies the patient as being at a risk for side effects.

16. The method of claim 15, wherein the method comprises determining the expression level of the XRCC1 gene and the IL-8 gene.

17. The method of claim 15, wherein the side effect is toxicity.

18. The method of claim 15, wherein the therapy comprises administration of at least one of a fluoropyrimidine drug and a platinum drug, or equivalent thereof.

19. The method of 18, wherein the fluoropyrimidine drug is 5-FU and the platinum drug is oxaliplatin.

20. The method of claim 15, wherein the suitable sample is at least one of a GI tumor sample, a sample of normal tissue corresponding to the GI tumor sample and a peripheral blood lymphocyte.

21. The method of claim 15, wherein the gastrointestinal cancer is selected from the group consisting of rectal cancer, colorectal cancer, metastatic colorectal cancer, colon cancer, gastric cancer, lung cancer, non-small cell lung cancer and esophageal cancer.

22. The method of claim 21, wherein the gastrointestinal cancer is colorectal cancer.


SEQUENCE LISTING (23 sequences)

SEQ ID NO: 1 - DNA, 17 bp, Artificial Sequence
  Description of Artificial Sequence Synthetic primer
gagcgcggctacagctt

SEQ ID NO: 2 - DNA, 22 bp, Artificial Sequence
  Description of Artificial Sequence Synthetic primer
tccttaatgtcacgcacgattt

SEQ ID NO: 3 - DNA, 18 bp, Artificial Sequence
  Description of Artificial Sequence Synthetic probe
accaccacggccgagcgg

SEQ ID NO: 4 - DNA, 22 bp, Artificial Sequence
  Description of Artificial Sequence Synthetic primer
gctcaacatgatgtttgcattc

SEQ ID NO: 5 - DNA, 18 bp, Artificial Sequence
  Description of Artificial Sequence Synthetic primer
gctggccctcgcttatga

SEQ ID NO: 6 - DNA, 24 bp, Artificial Sequence
  Description of Artificial Sequence Synthetic probe
tgcccagcacttcacgcatcagtt

SEQ ID NO: 7 - DNA, 24 bp, Artificial Sequence
  Description of Artificial Sequence Synthetic primer
cctgtaccagtccaataccatcct

SEQ ID NO: 8 - DNA, 20 bp, Artificial Sequence
  Description of Artificial Sequence Synthetic primer
tcctgctggtccttcccata

SEQ ID NO: 9 - DNA, 20 bp, Artificial Sequence
  Description of Artificial Sequence Synthetic probe
tcacctgggccgcacccttg

SEQ ID NO: 10 - DNA, 23 bp, Artificial Sequence
  Description of Artificial Sequence Synthetic primer
cagctctgtgtgaaggtgcagtt

SEQ ID NO: 11 - DNA, 24 bp, Artificial Sequence
  Description of Artificial Sequence Synthetic primer
gggtggaaaggtttggagtatgtc

SEQ ID NO: 12 - DNA, 35 bp, Artificial Sequence
  Description of Artificial Sequence Synthetic probe
tgcactgacatctaagttctttagcactccttggc

SEQ ID NO: 13 - DNA, 21 bp, Artificial Sequence
  Description of Artificial Sequence Synthetic primer
cctacgttgctggtctttctg

SEQ ID NO: 14 - DNA, 20 bp, Artificial Sequence
  Description of Artificial Sequence Synthetic primer
ctcctctggccctttctctg

SEQ ID NO: 15 - DNA, 34 bp, Artificial Sequence
  Description of Artificial Sequence Synthetic probe
ccacctggtatatgtcaaccttgtattctcaccc

SEQ ID NO: 16 - DNA, 17 bp, Artificial Sequence
  Description of Artificial Sequence Synthetic primer
cctgcggacggaatcct

SEQ ID NO: 17 - DNA, 19 bp, Artificial Sequence
  Description of Artificial Sequence Synthetic primer
gagtgagcagctggttcct

SEQ ID NO: 18 - DNA, 20 bp, Artificial Sequence
  Description of Artificial Sequence Synthetic primer
gctgtgatgagtggcaggct

SEQ ID NO: 19 - DNA, 19 bp, Artificial Sequence
  Description of Artificial Sequence Synthetic primer
tgatgagtggcaggctgtc

SEQ ID NO: 20 - DNA, 25 bp, Artificial Sequence
  Description of Artificial Sequence Synthetic probe
cagccagagatgtgacagccaccgt

SEQ ID NO: 21 - DNA, 20 bp, Artificial Sequence
  Description of Artificial Sequence Synthetic primer
ctgggaccgggtcaaaattg

SEQ ID NO: 22 - DNA, 23 bp, Artificial Sequence
  Description of Artificial Sequence Synthetic primer
ccgtacaaaactcaagccaaagg

SEQ ID NO: 23 - DNA, 25 bp, Artificial Sequence
  Description of Artificial Sequence Synthetic probe
tgcagccagccctacagcaaggact